(19) United States
(12) Patent Application Publication
(10) Pub. No.: US 2005/0042259 A1
(43) Pub. Date: Feb. 24, 2005

(54) Edible covering for livestock feedstuffs

(76) Inventor: Larry L. Berger, Champaign, IL (US)

Correspondence Address: GREER, BURNS & CRAIN, 300 S WACKER DR, 25TH FLOOR, CHICAGO, IL, 60606, US

(21) Appl. No.: 10/862,722

(22) Filed: Jun. 7, 2004

Related U.S. Application Data
(63) Continuation-in-part of application No. 10/198,866, filed on Jul. 19, 2002.

(51) Int. Cl. A61K 9/00; A23K 1/165

(52) U.S. Cl. 424/442; 426/635

(57) ABSTRACT

The instant invention is directed to a protective covering for application on livestock feedstuffs to prevent spoilage of the feedstuffs that is also edible by livestock, and includes a nutritive starch source to provide a nutritive aggregate ingredient, a salt to promote stability in a protective solution, a non-protein nitrogen source to act as a surfactant, and cement to promote spraying application of the protective solution formed from the covering. The instant invention is directed to a protective covering that includes a dry mixture, to which a sufficient amount of water is added to form an aqueous solution. The dry mixture includes a nutritive starch source, a salt source, a non-protein nitrogen source such as urea, and a gelling agent. Water is added to the dry mixture, resulting in the instant protective solution. The protective solution is then applied to various target surfaces, which include typical livestock feedstuffs such as silage, hay and grain. After it has set-up, the protective solution forms a protective covering over the feedstuffs.

### REFERENCE TO RELATED APPLICATION AND PRIORITY CLAIM

This application is a continuation-in-part of application Ser. No. 10/198,866, filed Jul. 19, 2002, and entitled Edible Covering for Livestock Feedstuffs. Priority is claimed from that application under 35 U.S.C. §120.

### FIELD OF THE INVENTION

The present invention relates generally to coverings for livestock feedstuffs and more particularly to edible protective coverings for livestock feedstuffs such as silage, hay and grain.

### BACKGROUND OF THE INVENTION

Weather damage and spoilage of stored livestock feedstuffs exposed to the elements has always been a concern for farmers. Spoilage losses of 15-30% are frequently observed when silages, hays or grains are exposed to the elements. For example, when rain or other precipitation falls on an exposed bale of hay, much of the rain is absorbed in the outer layers of the bale. The moisture content of the outer 10 to 20 centimeters of the hay is increased to between 25 and 40%. Mold and fungi that grow on the outer 10 to 20 centimeters make cattle and other livestock reluctant to consume this portion of the bales.

Barns and upright stave silos were built to minimize spoilage of stored feedstuffs. More recently, plastic sheets have been applied to hay bales and other stored feedstuffs to protect them from inclement weather and pests. Aside from problems posed by environmental disposal, the plastic sheets tend to trap moisture next to the hay, thereby promoting growth of molds and fungi. Moreover, if the livestock consume the plastic sheets along with the feedstuff underneath the plastic sheets, a blockage of the digestive tract may result in the animal's death.

Farmers have also covered bunker silos with plastic sheets held down by old vehicle tires. However, because the old vehicle tires are a breeding habitat for mosquitoes, many states, such as Illinois, have now made it illegal to use old tires for this purpose. Attempts at coating bales with tallow or other fats to provide a water barrier have also failed because during storage, which is typically anywhere from six to ten months, the fat becomes rancid and unpalatable so that the cattle may not eat either the fat or the feedstuff that it coated.

Attempts have also been made to cover bunker silos with substances such as lime, earth, candy, molasses and molasses-based products, small grain sod, such as wheat, Liqui-Seal manufactured by Cargill of Minneapolis, Minn., a product believed to be marketed under the name Nutri-Shield and believed to include the ingredients lignin sulfonate and molasses, sawdust, chopped straw, and composted manure solids. However, none of these methods have proven especially effective, and have even less benefit than plastic sheets in protecting the silage.

### SUMMARY OF THE INVENTION

The present invention is related to a protective covering for animal feedstuffs that is both edible by livestock that consume the feedstuffs and reduces spoilage of the feedstuffs due to exposure to the elements. The present invention includes a protective covering made from edible ingredients that may be turned into a solution and is then applied to stored animal feedstuffs to form a protective, water-resistant coating thereon. Application of this protective solution to the feedstuffs may be accomplished via a plurality of methods, such as spraying with a conventional commercial material sprayer. The instant invention reduces or obviates the need for plastic covers or buildings traditionally used to cover the feedstuffs during periods of inclement weather.

### DETAILED DESCRIPTION OF THE INVENTION

The preferred embodiment of the instant invention is a protective covering for application on livestock feedstuffs to prevent spoilage of the feedstuffs that is also edible by livestock, and includes a nutritive starch source to provide a nutritive aggregate ingredient, a salt to promote stability in a protective solution, a non-protein nitrogen source to act as a surfactant, and a gelling agent to promote spraying application of the protective solution formed from the covering. The instant invention includes a protective solution and a dry mixture to which a sufficient amount of water is added to form an aqueous solution. The dry mixture includes a nutritive starch source, a salt source, a non-protein nitrogen source such as urea, and a gelling agent such as calcium oxide, calcium hydroxide, or cement. Water is added to the dry mixture, resulting in the instant protective solution. The protective solution may then be applied to various target surfaces, which include typical livestock feedstuffs such as silage, hay and grain. After it has set-up, the protective solution forms a protective covering over the feedstuffs. Optionally, a layer of wax may subsequently be deposited over the protective coating for additional protection.

To create the dry mixture of the instant invention, the nutritive starch source may be selected from a variety of substances that are safely ingestible by livestock such as cattle, sheep, goats, pigs, poultry, and horses. Preferably the starch source includes a source of wheat, such as wheat grain or wheat flour that has been finely ground into particulate matter, since this is a nutritive starch source commonly consumed by livestock. Gluten proteins within the wheat are thought to form cross links with other gluten proteins, which form a starch/protein matrix having a rubber-like texture when semi-dry. Thus, the starch/protein matrix functions as an aggregate ingredient, similarly to the aggregate in concrete.

The salt source included in the preferred embodiment is sodium chloride (NaCl), although the instant invention contemplates additional salts such as potassium chloride (KCl), calcium chloride (CaCl), and magnesium chloride (MgCl₂). Typically, livestock have a natural appetite for sodium, and to a lesser extent, an appetite for potassium, calcium and magnesium. Thus, any of these salts may serve as the salt source, although NaCl is preferred. The inclusion of a salt source in the protective solution therefore serves both as a preservative and as an ingredient that will promote consumption of both the protective covering and the underlying feedstuffs by livestock. The preservative properties of salt have been well known for thousands of years. Indeed, when loose hay was stored in barns, it was historically common practice to salt the hay.

The non-protein nitrogen source is preferably urea, which, when ingested by ruminants such as cattle, degrades to ammonia. Ruminal bacteria incorporate the ammonia into bacterial protein, and the bacteria are then digested by cattle's small intestines, which become available to the ruminant in the lower digestive tract as a source of protein.

A gelling agent is also included in the protective solution so that when mixed with the other ingredients, the solution sets up like concrete, but is also edible by livestock. The instant invention contemplates that a plurality of gelling agents may be used, such as calcium oxide, calcium hydroxide or cement. Because it is consumed by the livestock as part of the protective covering, the preferred gelling agent, however, comports with relevant FDA and American Feed Control Official (AFCO) guidelines, which specifically include calcium oxide and calcium hydroxide. The gelling agent preferably exhibits properties that make the protective covering more susceptible of widespread application. For example, addition of calcium hydroxide, calcium oxide or cement makes the protective covering less viscous while also allowing the mixture to set-up more quickly following application. While the instant invention contemplates use of numerous commercial cements, a preferred embodiment includes #1 Portland Cement, commercially available from numerous sources. Similarly, the instant invention contemplates use of any commercially available source of pure CaO or CaOH.

Each of the ingredients of the dry mixture is added in a predetermined dry weight. Thus, the combined dry weights of the nutritive starch source, the salt, the urea and the gelling agent have a dry mixture weight, to which a sufficient weight of water is added to liquefy the protective solution. Generally, the amount of water added by weight should equal a predetermined percentage of the total combined dry mixture weight of the dry mixture ingredients. Preferably, a weight of water approximately equal to between 40% and 100% of the dry mixture weight is added to form the protective solution. Provided one anticipates the approximate dry mixture weight, the water may be added to any combination of the dry ingredients or to a single dry ingredient in any order, even before the dry ingredients are combined with one another.

Predetermined amounts of the protective solution ingredients are added to the protective solution so that the resulting solution exhibits a viscosity susceptible of fluid application. In the preferred embodiment of the instant invention, predetermined amounts of the protective solution ingredients are determined using the percentage by weight of the total protective solution. Optimally, the nutritive starch source is provided in a range of 25-75% of the dry weight, the salt is provided in a range of 25-75% of the dry weight, the urea is provided in a range of 2-10% of the dry weight, and the gelling agent is provided in a range between 2-10% of the dry weight. While the starch source to salt ratio should be sufficient to prevent attack of the starch/salt matrix by yeast, bacteria or molds, the percentages of the wheat and salt may be varied without altering the essence of the invention.

One preferred dry mixture includes 45% ground wheat by dry weight, 45% NaCl by dry weight, 5% urea by dry weight, and 5% gelling agent by dry weight. The total dry weight is then calculated. An amount of water is then added to the dry mixture that is equal in weight to a predetermined percentage of the total dry weight of the dry mixture, resulting in an aqueous solution. As much as 100% of the dry mixture weight of water has been added to the dry mixture. However, adding significantly less amounts of water to the dry mixture will not compromise or adversely affect the properties of the resulting protective solution. As little as 40% of the dry mixture weight of water has successfully been added to the dry mixture without compromising the effectiveness of the protective solution. Water of any temperature is contemplated for use with the instant invention, preferably in the range of 33 to 212° Fahrenheit. Moreover, the water may be tap water, and requires no pre-treatment prior to its addition to the dry mixture.

The instant invention contemplates numerous variations of the percentages by weight of each ingredient, depending only on the farmer or other operator's particular application needs. For example, a preferred method for application of the protective solution to the target surfaces is a spraying application whereby an operator may utilize a typical commercial material sprayer, such as that obtained from Reed Concrete Pumps, 13822 Oaks Avenue, Chino, Calif. 91710, to spray the target surface with protective solution until the protective solution forms a covering of a desired predetermined thickness. The thickness of the covering sprayed on the target surfaces range from approximately 0.25 inch to 1.0, with an optimal range of between 0.4 inch and 0.6 inch, and is preferably approximately 0.5 inch. However, the instant invention contemplates even a wider range of thicknesses for the protective covering, depending on the topography of the target surface. For example, if the target surface is relatively smooth, lesser thicknesses are adequate. If the target surface is relatively rough and uneven, greater thicknesses are contemplated. Once a coating of protective solution has been applied to the target surfaces via spraying, the protective solution will harden and set, forming a protective covering over the target surface.

Thus, the instant protective solution contemplates the addition various amounts of water to the dry mixture, depending on the needs of the user. Consequently, decreasing the amount of water added to the dry mixture allows the user to vary the amounts of other ingredients as well. For example, because the urea acts similarly to a surfactant, the relative amount of urea affects the relative amount of water that may be used in the overall protective solution. Typically, a surfactant allows a liquid to coat particulate matter with a thinner sheet of liquid. Therefore, the more urea included in the protective solution, the less the amount of water is generally needed. Thus, the addition of urea to the protective solution not only serves as a crude protein source, but reduces the amount of water necessary to obtain good handling properties of the protective solution. Reducing the water to gelling agent ratio allows the protective solution to “set up” more quickly. Moreover, because less water increases the overall viscosity of the protective solution, the protective solution is more likely to remain where it is deposited, reducing run-off of the protective solution once deposited. This is especially advantageous when the target surface is relatively rough and uneven, wherein aqueous substances would otherwise tend to seek a lower altitudinal surface rather than being evenly distributed over the surface.

Optionally, additional ingredients may be added to the invention without diminishing the benefits of the core protective solution. For example, one optional substance that may be added to the dry mixture of the instant invention is an oil, such as soybean oil, to keep the aqueous protective solution pliable. Soybean oil is not necessary to the preferred embodiment of the instant invention.

Yet another optional substance that may be added to the dry mixture of the instant invention is potassium bitartrate, or cream of tartar, which acts as a leavening agent. However, like soybean oil, this optional substance is not necessary to the preferred embodiment of the instant invention.

Another optional substance that may be deposited on the livestock feedstuffs following application of the protective solution is a paraffin or wax emulsion, such as the type of wax emulsion typically sprayed on boxes that serve as shipping containers for meat and vegetables. Once applied, this wax emulsion helps prevent moisture penetration during periods of extended drizzle or high humidity. Animals consume the wax emulsion in combination with the protective covering and feedstuff without concern. Indeed, many commercial chocolates consumed by humans contain a similar wax emulsion. The wax emulsion may be deposited in layers having a variety of thicknesses sufficient to seal the starch-salt matrix to prevent water penetration, with an optimum thickness range of between 0.001 and 0.1 inches. Alternatively, it is contemplated by the instant invention that a layer of wax paper could be deposited atop the protective solution to prevent moisture penetration as well.

Embodiments of the instant invention additionally contemplate the addition of wax to the dry solution or to the protective solution as a constituent part of the respective dry solution or protective solution. The addition of the wax to the dry solution or protective solution is advantageous in that the additional step of depositing wax atop the set up protective solution is eliminated, and instead accomplished during the initial deposition of the protective solution. It is believed that from between approximately 5% and 10% by weight of wax emulsion is adequate for embodiments that include wax emulsion as a constituent of either the dry solution or protective solution. It is further believed that the following the addition of the wax emulsion, volatile agents could be permitted to evaporate so that the wax emulsion becomes part of a solid mixture.

Still another embodiment of the instant invention includes the addition of a predetermined amount of magnesium oxide (MgO) to the dry mixture. The amount of MgO to be added by dry weight to the protective covering is roughly equal to a combined amount of nutritive starch source and salt source that is removed. MgO is not soluble in water, and is a nutrient source that is added to many diets, including those of cattle and other livestock. In laboratory studies where “drip tests” were conducted, protective solution that included MgO exhibited reduced erosion of the starch/salt matrix. A “drip-test” is performed by using a separatory funnel and placing it approximately 15 inches above the feedstuff that has been coated with the protective solution and allowed to harden into a protective covering. A layer of wax emulsion was added following the deposition of the protective solution. Water is then dripped onto a given location continuously for a predetermined number of hours. The “crater” formed as a result of the dripping water is then measured. It was found that if and when the wax emulsion overlying the protective covering were cracked, the MgO provided additional durability to the starch/salt matrix so that it was more stable.

The protective covering formed by the protective solution of the instant invention exhibits a lifespan that is believed to be indefinitely long, dependent only on the surrounding elemental conditions. Experimental observations of feedstuffs following deposition of the protective solution and wax emulsion have revealed that the protective covering has remained in tact for periods of one year. Based on these experimental results, the inventors project that the protective covering may be effective for many years following deposition of the protective solution.

While the preferred method for application of the protective solution to the target surfaces is a spraying application utilizing a typical commercial material sprayer, embodiments of the instant invention contemplate a variety of methods for application of the protective solution, and when included, the wax layer as well. For example, the protective coating may be applied as a pumped slurry, either mechanically or hand sprayed, or applied via a paint roller or a rotating brush, to name a few examples. It is also contemplated that additional methods of application may proceed via spraying of the livestock feedstuffs with a hose that is fluidly coupled to a source of the protective solution, or via application with a boom sprayer that is fluidly coupled to a source of the protective solution.

In one embodiment, it is contemplated that a boom sprayer may deposit the protective solution, and when, following a predetermined period of time, the protective solution has set up, a wax coating may subsequently be applied via boom sprayer. For example, a single boom sprayer having two booms may be used, wherein the first boom emits and deposits the protective solution, and the second boom follows the first, emitting and depositing the layer of wax atop the protective solution.

While particular embodiments of the protective covering of the present invention has been described herein, it will be appreciated by those skilled in the art that changes and modifications may be made thereto without departing from the invention in its broader aspects and as set forth in the following claims.

1. A protective covering for livestock feedstuffs to prevent spoilage of the livestock feedstuffs that is also edible by livestock comprising:
a predetermined amount of a nutritive starch source;
a predetermined amount of a salt suitable for ingestion by livestock;
a predetermined amount of a non-protein nitrogen source; and
a predetermined amount of a gelling agent.

2. The protective covering of claim 1 further comprising a predetermined amount of water.

3. The protective covering of claim 2 wherein said predetermined amounts of said nutritive starch source, said salt, said urea and said gelling agent have a combined weight, and said predetermined amount of water comprises an amount by weight approximately equal to a predetermined percentage of said combined weight.

4. The protective covering of claim 3 wherein said predetermined percentage comprises between 40% and 100% of said combined weight.

5. The protective covering of claim 1 wherein said nutritive starch source is wheat flour that has been finely ground into particulate matter.

6. The protective covering of claim 1 wherein said nutritive starch source is wheat grain that has been finely ground into particulate matter.

7. The protective covering of claim 1 wherein said predetermined amount of said nutritive starch source comprises approximately 45% of said covering by dry weight.

8. The protective covering of claim 1 wherein said salt is selected from the group consisting of NaCl, KCl, CaCl, and MgCl₂.

9. The protective covering of claim 1 wherein said predetermined amount of said salt comprises approximately 45% of said covering by dry weight.

10. The protective covering of claim 1 wherein said predetermined amount of said non-protein nitrogen source comprises a predetermined amount of urea.

11. The protective covering of claim 10 wherein said predetermined amount of said urea comprises approximately 5% of said covering by dry weight.

12. The protective covering of claim 1 wherein said gelling agent is selected from the group consisting of calcium oxide and calcium hydroxide and cement.

13. The protective covering of claim 1 wherein said predetermined amount of gelling agent comprises approximately 5% of said covering by dry weight.

14. The protective covering of claim 1 wherein said protective covering further comprises a predetermined amount of magnesium oxide.

15. The protective covering of claim 14 wherein a predetermined combined amount of said nutritive starch source and said salt is removed and an equal amount by dry weight of said magnesium oxide is added to said protective covering.

16. The protective covering of claim 1 wherein said protective covering is formed into a coating between 0.25 inch and 1.0 inch thick.

17. The protective covering of claim 1 further comprising a wax agent.

18. The protective covering of claim 17 wherein said wax agent is selected from the group consisting of a wax emulsion and paraffin.

19. A protective covering for livestock feedstuffs that is also edible by livestock comprising:
nine parts per weight of a wheat source;
nine parts per weight of a salt source;
one part per weight of urea; and
one part per weight of gelling agent.

20. The protective covering of claim 19 further comprising from between 4 and 10 parts per weight of water.

21. The protective covering of claim 19 wherein said salt source is selected from the group consisting of NaCl, KCl, CaCl, and MgCl₂.

22. A method for providing an edible covering for livestock feedstuffs such as hay, silage and grain to protect the feedstuffs from spoilage due to precipitation, fungal, mold and bacterial growth comprising, said method comprising the steps of:
selecting a gelling agent from the group consisting of calcium oxide and calcium hydroxide;
preparing a dry mixture that includes the gelling agent and is capable of forming a protective coating that is also suitable for ingestion by livestock;
adding a predetermined amount of water to said dry mixture sufficient to form a protective solution;
applying said protective solution to livestock feedstuffs; and
forming a protective coating having a predetermined thickness on the livestock feedstuffs.

23. The method of claim 22 wherein said step of applying said protective solution comprises spraying a predetermined amount of said protective solution on the livestock feedstuffs.

24. The method of claim 22 wherein the protective solution is painted onto the livestock feedstuffs via a paint roller or paint brush.

25. The method of claim 22 wherein said step of applying said protective solution comprises pumping a predetermined amount of said protective solution on the livestock feedstuffs.

26. The method of claim 22 wherein said step of applying said protective solution comprises spraying the livestock feedstuffs with a hose that is fluidly coupled to a source of said protective solution.

27. The method of claim 22 wherein said step of applying said protective solution comprises application with a boom sprayer that is fluidly coupled to a source of said protective solution.

28. The method of claim 22 wherein said step of forming a protective coating comprises allowing said protective solution to set up on the livestock feedstuffs.

29. The method of claim 22 wherein said step of adding a predetermined amount of water comprises measuring a dry weight of said dry mixture and mixing into said dry mixture an amount of water equal in weight to a predetermined percentage of said dry weight of said dry mixture.

30. The method of claim 29 wherein said predetermined percentage is between approximately 40% and 100% of said dry weight of said dry mixture.

31. The method of claim 22 wherein said method further includes a step of applying a wax emulsion on the livestock feedstocks following said forming a protective coating.

32 The method of claim 31 wherein said step of applying a wax emulsion is applied to have a thickness of approximately 0.001 and 0.1 inches.

33. The method of claim 22 wherein said protective coating is formed to be between 0.25 inch and 1.0 inch thick.

34. A protective covering for application on livestock feedstuffs to prevent spoilage that is also edible by livestock comprising:
a nutritive starch source to provide a nutritive aggregate ingredient;
a salt to promote stability in said protective solution;
a non-protein nitrogen source to act as a surfactant; and
a gelling agent to promote spraying application of said protective solution.

35. The protective covering of claim 34 wherein said salt and said nutritive starch source form a starch/salt matrix.

36. The protective covering of claim 35 wherein said salt and said nutritive starch source are provided in a predetermined ratio to prevent erosion of said starch/salt matrix.

37. The protective covering of claim 35 further comprising magnesium oxide to prevent erosion of said starch/salt matrix.

38. The protective covering of claim 34 further comprising a wax emulsion for providing a moisture barrier over said protective solution.

39. The protective covering of claim 34 further comprising an erosion inhibitor.

40. The protective covering of claim 39 wherein said erosion inhibitor is MgO.

41. A protective covering for application on livestock feedstuffs to prevent spoilage that is also edible by livestock comprising:
a nutritive starch source;
a salt source;
urea;
a gelling agent; and
water;
wherein said nutritive starch source, said salt source, said urea, said gelling agent and said water are provided in ratios that form a liquid substances that dries into a solid when applied in thicknesses of between 0.25 inch and 1.0 inch thick.
